(19) United States
(12) Patent Application Publication
(10) Pub. No.: US 2026/0325589 A1
(43) Pub. Date: Oct. 22, 2026

(54) COMPOSITION FOR IMPROVING SKIN FUNCTION COMPRISING ENTEROCOCCUS FAECALIS AS AN ACTIVE INGREDIENT

(71) Applicant: BEREUM CO.,LTD, Wonju-si (KR)

(72) Inventors: Kwon Il HAN, Wonju-Si (KR); Hyun Dong SHIN, Wonju-Si (KR); Eun Jung MOON, Wonju-Si (KR); Yu Ra LEE, Wonju-Si (KR); Youn Bum PARK, Wonju-Si (KR); Sun Hwa BAEK, Wonju-Si (KR); Pan Soo KIM, Suwon-Si (KR); Doo Sin JO, Daegu (KR)

(73) Assignee: BEREUM CO.,LTD, Wonju-si (KR)

(21) Appl. No.: 19/722,687

(22) Filed: Jun. 26, 2026

Related U.S. Application Data
(63) Continuation of application No. PCT/KR2024/020967, filed on Dec. 23, 2024.

(30) Foreign Application Priority Data
Dec. 29, 2023 (KR) 10-2023-0196260

(51) Int. Cl. A61K 35/744; A61K 8/99; A61P 17/00; A61Q 19/00; A61Q 19/02; A61Q 19/08

(52) U.S. Cl. CPC A61K 35/744; A61K 8/99; A61P 17/00; A61Q 19/00; A61Q 19/005; A61Q 19/02; A61Q 19/08; A61K 2800/74

(57) ABSTRACT

The present invention relates to a composition for improving skin function comprising Enterococcus faecalis as an active ingredient, and more particularly, to a composition for skin whitening, improvement of skin barrier function, improvement of skin wrinkles, enhancement of skin elasticity, or skin soothing, comprising, as an active ingredient, an Enterococcus faecalis EF-2001 strain, a lysate thereof, a culture thereof, a concentrate thereof, a dried product thereof, or dead cells thereof.

### CROSS-REFERENCE TO RELATED APPLICATION

This application is a Bypass Continuation of International Application No. PCT/KR2024/020967, filed on Dec.23, 2024, which claims benefit of priority from the prior Korean Patent Application No. 10-2023-0196260, filed on Dec. 29, 2023, the disclosure of which is incorporated herein by reference in its entirety.

### TECHNICAL FIELD

The present invention relates to a composition for improving skin function comprising Enterococcus faecalis as an active ingredient, and more particularly, to a composition for skin whitening, improving skin barrier function, improving skin wrinkles, enhancing skin elasticity, or soothing skin, comprising, as an active ingredient, an Enterococcus faecalis EF-2001 strain, a lysate thereof, a culture thereof, a concentrate thereof, a dried product thereof, or dead cells thereof.

### BACKGROUND ART

The skin is an organ located at the outermost part of the human body, and performs a role of maintaining homeostasis and protecting the body by sensing various external stimuli. In addition, the skin is known to function as an immune organ that prevents foreign substances from entering from the outside. Among the constituent layers of the skin, the epidermis serves to prevent moisture loss from inside the human body, and among the four layers of the epidermis, namely the stratum corneum, granular layer, spinous layer, and basal layer, intercellular lipids between keratinocytes of the stratum corneum form a skin barrier. It is known that phenomena such as deterioration of normal skin function, loss of elasticity, wrinkle formation, and excessive melanin production are promoted by external stimuli such as ultraviolet rays and fine dust, and internal factors such as metabolites produced by intestinal microorganisms; however, there are few solutions capable of controlling such phenomena.

In this regard, primary cilia are antenna-like structures protruding from the cell surface, and are non-motile organelles present in almost all eukaryotic cells, including skin cells. Such primary cilia sense external stimuli or various sensory signals, such as visual, olfactory, auditory, and motor sensory signals, and mediate signaling pathways that are crucial for maintaining tissue homeostasis in vivo, such as Hedgehog (Hh), transforming growth factor-β (TGF-β), platelet-derived growth factor (PDGF), AMP-activated protein kinase (AMPK), and autophagy.

In particular, according to recent studies, primary cilia activity has been reported to be essential for growth and differentiation of skin cells, strengthening of the skin barrier, prevention of excessive melanin production, and maintenance of tissue homeostasis of the skin epidermis.

Accordingly, research and development on materials for activating such primary cilia remain insufficient. Further, in the cosmetics field, it is important that a material does not cause toxicity or irritation to the skin, and thus research using natural substances for activating primary cilia is required.

In this regard, microorganisms belonging to the genus Enterococcus are widely present in nature and aerobically utilize carbohydrates. In general, bacteria such as microorganisms belonging to the genus Enterococcus are known to prevent damage caused by pathogenic microorganisms through antagonistic action in vivo or secreted antimicrobial substances. Among them, Enterococcus faecalis EF-2001 was identified through screening of the intestinal microflora of a two-year-old girl. A dead cell product of Enterococcus faecalis EF-2001 is obtained by heat-treating and killing Enterococcus faecalis EF-2001 and recovering cell components thereof.

According to studies reporting the physiological activity of Enterococcus faecalis EF-2001, in mice suffering from DSS (dextran sulfate sodium)-induced colitis, mice that ingested Enterococcus faecalis EF-2001 exhibited a DSS alleviating effect, and it was reported that proliferation of transplanted sarcoma cells (Sarcoma-180) was reduced and NK cells were activated (Tadano et al., J. Japan Mibyou System Association, 2011). In addition, with respect to inhibition of harmful bacteria and intestinal regulation, Enterococcus faecalis EF-2001 exhibits an effect of improving and preventing symptoms by inhibiting the activity of Candida albican, which is a cause of tongue coating (Ishijima et al., Med. Mycol. J, 2014), and it has been disclosed that, in antibiotic-administered mice, beneficial bacteria rapidly proliferate and harmful bacteria are inhibited as compared with an intestinal control group (Simohashi et al., Medicine and Biology, 2002).

Meanwhile, a dead cell product of Enterococcus faecalis EF-2001 exhibits various physiological activities, which is based on the fact that, due to the characteristics of dead cells, it is not affected by heat and pH and can be processed into various types of formulations (Kan, Food Industry, 2001). In addition, Enterococcus faecalis EF-2001 has a bacterial cell content of 7.5 trillion cells per gram, and thus a large amount of lactic acid bacterial cells can be ingested even in a small amount.

However, specific effects of such Enterococcus faecalis on improving skin function, namely inhibition of melanin production, enhancement of skin elasticity, improvement of skin barrier function, soothing of the skin, and the like, have not been specifically known.

Under these circumstances, the present inventors made efforts to develop a material capable of improving overall skin functions, such as controlling excessive melanin production, enhancing skin elasticity, reducing inflammatory responses, and improving the skin barrier, by activating primary cilia. As a result, the present inventors confirmed that Enterococcus faecalis EF-2001, a lysate thereof, a culture thereof, a concentrate thereof, a dried product thereof, and dead cells thereof are effective in improving overall skin functions, such as controlling excessive melanin production, enhancing skin elasticity, reducing inflammatory responses, and improving the skin barrier, by activating primary cilia, thereby completing the present invention.

### Patent Documents

Patent Document 0001 Korean Patent Registration No. 10-2503163

Patent Document 0002 Korean Patent Application Publication No. 10-2022-0079092

### DISCLOSURE

### Technical Problem

An object of the present invention is to provide a composition for improving skin function, comprising, as an active ingredient, at least one selected from the group consisting of an Enterococcus faecalis strain, a lysate thereof, a culture thereof, a concentrate thereof, a dried product thereof, and dead cells thereof.

Another object of the present invention is to provide a composition for improving skin function, which inhibits excessive melanin production, enhances skin elasticity, improves skin barrier function, and has a skin soothing effect by activating primary cilia.

Another object of the present invention is to provide a cosmetic product for improving skin function, comprising, as an active ingredient, at least one selected from the group consisting of an Enterococcus faecalis strain, a lysate thereof, a culture thereof, a concentrate thereof, a dried product thereof, and dead cells thereof.

Another object of the present invention is to provide a health functional food for improving skin function, comprising, as an active ingredient, at least one selected from the group consisting of an Enterococcus faecalis strain, a lysate thereof, a culture thereof, a concentrate thereof, a dried product thereof, and dead cells thereof.

Another object of the present invention is to provide a food additive for improving skin function, comprising, as an active ingredient, at least one selected from the group consisting of an Enterococcus faecalis strain, a lysate thereof, a culture thereof, a concentrate thereof, a dried product thereof, and dead cells thereof.

Another object of the present invention is to provide an external preparation for improving skin function, comprising, as an active ingredient, at least one selected from the group consisting of an Enterococcus faecalis strain, a lysate thereof, a culture thereof, a concentrate thereof, a dried product thereof, and dead cells thereof.

Another object of the present invention is to provide a pharmaceutical composition for improving skin function, comprising, as an active ingredient, at least one selected from the group consisting of an Enterococcus faecalis strain, a lysate thereof, a culture thereof, a concentrate thereof, a dried product thereof, and dead cells thereof.

### Technical Solution

In order to achieve the above objects, the present invention provides a composition for improving skin function, comprising, as an active ingredient, at least one selected from the group consisting of an Enterococcus faecalis strain, a lysate thereof, a culture thereof, a concentrate thereof, a dried product thereof, and dead cells thereof.

In addition, the present invention provides the composition for improving skin function, wherein the strain is an Enterococcus faecalis EF-2001strain.

In addition, the present invention provides the composition for improving skin function, wherein the composition activates primary cilia.

In addition, the present invention provides the composition for improving skin function, wherein the composition inhibits melanin production.

In addition, the present invention provides the composition for improving skin function, wherein the composition enhances skin elasticity.

In addition, the present invention provides the composition for improving skin function, wherein the composition has activity of improving skin barrier function.

In addition, the present invention provides the composition for improving skin function, wherein the composition has a skin soothing effect.

In addition, the present invention provides a cosmetic product for improving skin function, comprising, as an active ingredient, at least one selected from the group consisting of an Enterococcus faecalis strain, a lysate thereof, a culture thereof, a concentrate thereof, a dried product thereof, and dead cells thereof.

In addition, the present invention provides a health functional food for improving skin function, comprising, as an active ingredient, at least one selected from the group consisting of an Enterococcus faecalis strain, a lysate thereof, a culture thereof, a concentrate thereof, a dried product thereof, and dead cells thereof.

In addition, the present invention provides a food additive for improving skin function, comprising, as an active ingredient, at least one selected from the group consisting of an Enterococcus faecalis strain, a lysate thereof, a culture thereof, a concentrate thereof, a dried product thereof, and dead cells thereof.

In addition, the present invention provides an external preparation for improving skin function, comprising, as an active ingredient, at least one selected from the group consisting of an Enterococcus faecalis strain, a lysate thereof, a culture thereof, a concentrate thereof, a dried product thereof, and dead cells thereof.

In addition, the present invention provides a pharmaceutical composition for improving skin function, comprising, as an active ingredient, at least one selected from the group consisting of an Enterococcus faecalis strain, a lysate thereof, a culture thereof, a concentrate thereof, a dried product thereof, and dead cells thereof.

### Advantageous Effect

The composition for improving skin function according to the present invention, comprising, as an active ingredient, at least one selected from the group consisting of an Enterococcus faecalis strain, a lysate thereof, a culture thereof, a concentrate thereof, a dried product thereof, and dead cells thereof, has a skin whitening effect by inhibiting melanin production through activation of primary cilia, improves skin barrier function by improving expression of Claudin-1 protein, and exhibits skin wrinkle improvement and soothing effects by inhibiting expression of MMP-1, which is a collagenase, and inhibiting activation of nuclear factor-κB (NF-κB).

Therefore, the composition for improving skin function may be usefully used in a cosmetic product, a health functional food, a food additive, an external preparation, and a pharmaceutical composition for improving skin function.

### DESCRIPTION OF DRAWINGS

FIG. 1 shows the number of primary cilia-forming cells and the length of primary cilia by treatment with a culture solution and dead cells of an Enterococcus faecalis EF-2001 strain according to Experimental Example 1 of the present invention.

FIG. 2 shows the number of primary cilia-forming cells and the length of primary cilia when cells treated with a stress-inducing substance are treated with dead cells of an Enterococcus faecalis EF-2001 strain according to Experimental Example 1 of the present invention.

FIG. 3 shows measurement of melanin content by treatment with a culture solution and dead cells of an Enterococcus faecalis EF-2001 strain according to Experimental Example 2 of the present invention.

FIG. 4 is an image showing Claudin-1 fluorescence expression and skin barrier formation by treatment with dead cells of an Enterococcus faecalis EF-2001 strain according to Experimental Example 3 of the present invention.

FIG. 5 shows expression of Claudin-1 protein by treatment with dead cells of an Enterococcus faecalis EF-2001 strain according to Experimental Example 3 of the present invention.

FIG. 6 shows expression of MMP-1 by treatment with dead cells of an Enterococcus faecalis EF-2001 strain according to Experimental Example 4 of the present invention.

FIG. 7 shows activation of NF-κB by treatment with dead cells of an Enterococcus faecalis EF-2001 strain according to Experimental Example 4 of the present invention.

### MODE FOR INVENTION

Hereinafter, preferred embodiments of the present invention will be described in detail so that a person having ordinary skill in the art to which the present invention pertains can easily carry out the present invention.

Various embodiments described herein are described with reference to the drawings. In the following description, various specific details, such as specific forms, compositions, processes, and the like, are set forth in order to provide a thorough understanding of the present invention. However, certain embodiments may be practiced without one or more of these specific details, or together with other known methods and forms. In other instances, known processes and manufacturing techniques are not described in specific detail so as not to unnecessarily obscure the present invention. Reference throughout the present specification to “one embodiment” or “an embodiment” means that a particular feature, form, composition, or characteristic described in connection with the embodiment is included in one or more embodiments of the present invention. Accordingly, the appearances of “one embodiment” or “an embodiment” in various places throughout the present specification do not necessarily refer to the same embodiment of the present invention. In addition, particular features, forms, compositions, or characteristics may be combined in any suitable manner in one or more embodiments.

Unless otherwise defined herein, all scientific and technical terms used herein have the same meanings as commonly understood by a person skilled in the art to which the present invention pertains.

In one embodiment, the present invention provides a composition for improving skin function, comprising, as an active ingredient, at least one selected from the group consisting of an Enterococcus faecalis strain, a lysate thereof, a culture thereof, a concentrate thereof, a dried product thereof, and dead cells thereof. The Enterococcus faecalis strain is preferably one registered as Enterococcus faecalis EF-2001 (Korean Accession No.: KCTC SD1560), but is not limited thereto.

As used herein, the term “lysate” means a product obtained by lysing the Enterococcus faecalis strain of the present invention by a known method.

As used herein, the term “culture” means growing the Enterococcus faecalis strain of the present invention under appropriately controlled environmental conditions. The culturing process of the present invention may be performed according to an appropriate medium and culture conditions known in the art. Such a culturing process may be easily adjusted and used by a person skilled in the art depending on the selected strain. Specifically, the culture may be batch culture, continuous culture, or fed-batch culture, but is not limited thereto.

For example, the Enterococcus faecalis strain of the present invention may be cultured in a nutrient medium (NA medium) under one or more conditions selected from a temperature of 25°C to 40°C, a stirring speed of 50 rpm to 200 rpm, and a pH of 6 to 9, but is not limited thereto. In addition, the culturing may be performed under a relative humidity condition of 40% to 50% based on relative humidity for 24 hours, or under a temperature condition of 30°C to 40°C, but is not limited thereto.

As used herein, the term “culture solution” means a culture medium obtained by culturing the Enterococcus faecalis strain, and may be a culture medium containing the strain or a cell-free culture medium from which the bacterial cells have been removed.

In one specific embodiment of the present invention, the culture may be a culture medium obtained after culturing an Enterococcus faecalis EF-2001strain in a liquid culture medium. Specifically, the culture solution of the Enterococcus faecalis EF-2001strain may be an original culture solution containing bacterial cells, and may also be bacterial cells obtained by removing or concentrating the culture supernatant. The composition of the culture solution may further include not only components necessary for ordinary culture of Enterococcus, but also components that act synergistically on the growth of Enterococcus, and the composition thereof may be easily selected by a person having ordinary skill in the art. In addition, the form of the culture may be liquid or solid, and is not limited to any one thereof.

As used herein, the term “concentrate” means a product obtained by concentrating the culture of the strain by a conventional method. The concentrate may be a concentrate obtained by concentrating, by a conventional method, a liquid culture medium in which the Enterococcus faecalis EF-2001 strain has been cultured; however, the form of the culture is not limited to a liquid.

As used herein, the term “dried product” or “dried product of a culture” means a product obtained by drying the culture of the strain by a conventional method. The dried product of the culture may be a dried concentrate obtained by drying, by a conventional method, a liquid culture medium in which the Enterococcus faecalis EF-2001strain has been cultured; however, the form of the culture is not limited to a liquid.

In addition, in the present invention, as the Enterococcus faecalis strain or dead cells thereof, any commercially available product or any product prepared by a known method for preparing dead cells may be used, and the same does not exhibit toxicity and is harmless to the human body.

In addition, the dead cells may be prepared by heat-treating corresponding live cells or treating the same with formalin or another sterilizing agent, and the dead cells may be used even when they are substantially dead.

In addition, examples of the dead cells include a dead cell suspension or a dried product thereof, which is obtained by washing a strain obtained by culturing according to a conventional method, centrifugally dehydrating the strain, repeating washing and dehydration as necessary, suspending the strain in distilled water, physiological saline, or the like, and heating the suspension, for example, at 80°C to 115°C for 30 minutes to 3 seconds; or a dead cell suspension or a dried product thereof obtained by irradiating the dead cell suspension with gamma rays or neutron rays.

As a drying means for the dead cell suspension, any known drying means may be used without particular limitation, and examples thereof include spray drying and freeze drying. In some cases, before or after sterilization treatment by heating or the like, or before or after drying treatment, enzyme treatment, surfactant treatment, or grinding and pulverization treatment may be performed, and products obtained by such treatments are also included in the dead cells of the present invention.

In addition, the dead cells may be prepared by the following method, but are not limited thereto:
i) seed-culturing Enterococcus faecalis, and then main-culturing the Enterococcus faecalis at a pH of 4.0 to 9.0 and a temperature of 15°C to 45°C, more preferably at a pH of 5.0 to 8.0 and a temperature of 20°C to 40°C; and
ii) heat-treating the Enterococcus faecalis main-cultured in step 1) at a temperature of 60°C to 140°C for 1 minute to 40 minutes, more preferably at a temperature of 70°C to 130°C for 5 minutes to 30 minutes, followed by drying and powdering.

In another embodiment of the present invention, the composition for improving skin function may have an effect of activating primary cilia.

In this regard, primary cilia are antenna-like structures protruding from the cell surface, and are non-motile organelles present in almost all eukaryotic cells, including skin cells. Such primary cilia sense external stimuli or various sensory signals, such as visual, olfactory, auditory, and motor sensory signals, and mediate signaling pathways that are crucial for maintaining tissue homeostasis in vivo, such as Hedgehog (Hh), transforming growth factor-β (TGF-β), platelet-derived growth factor (PDGF), AMP-activated protein kinase (AMPK), and autophagy.

According to recent studies, primary cilia activity has been reported to be essential for growth and differentiation of skin cells, strengthening of the skin barrier, prevention of excessive melanin production, and maintenance of tissue homeostasis of the skin epidermis. Accordingly, the composition for improving skin function of the present invention can achieve the intended effects of the present invention by activating primary cilia.

In addition, according to one embodiment of the present invention, activation of the primary cilia can be confirmed by measuring a primary cilia formation ratio and the length of primary cilia.

In another embodiment of the present invention, the composition for improving skin function may have an effect of inhibiting melanin production.

In another embodiment of the present invention, the composition for improving skin function may have an effect of enhancing skin elasticity.

In another embodiment of the present invention, the composition for improving skin function may have activity of improving skin barrier function.

In another embodiment of the present invention, the composition for improving skin function may have a skin soothing effect.

In order to stably preserve the Enterococcus faecalis EF-2001 strain of the present invention for a long period of time, bacterial cells may be suspended in a storage solution prepared by mixing a predetermined amount of glycerol with water and stored at -70°C, or may be suspended in sterilized 10% skim milk and freeze-dried. However, the preservation method is not limited thereto, and the strain may be made into a long-term preservation form by various known methods.

Meanwhile, the concentration of the Enterococcus faecalis EF-2001strain in the composition is not particularly limited, but may be 10³ CFU/ml to 10¹⁰ CFU/ml, 10³ CFU/ml to 10⁹ CFU/ml, 10³ CFU/ml to 10⁸ CFU/ml, 10³ CFU/ml to 10⁷ CFU/ml, 10³ CFU/ml to 10⁶ CFU/ml, 10³ CFU/ml to 10⁵ CFU/ml, 10³ CFU/ml to 10⁴ CFU/ml, 10⁴ CFU/ml to 10¹⁰ CFU/ml, 10⁵ CFU/ml to 10¹⁰ CFU/ml, 10⁶ CFU/ml to 10¹⁰ CFU/ml, 10⁷ CFU/ml to 10¹⁰ CFU/ml, 10⁸ CFU/ml to 10¹⁰ CFU/ml, 10⁹ CFU/ml to 10¹⁰ CFU/ml, 10⁴ CFU/ml to 10⁹ CFU/ml, 10⁵ CFU/ml to 10⁸ CFU/ml, or 10⁶ CFU/ml to 10⁷ CFU/ml.

The composition may further include a cryoprotectant or an excipient. The cryoprotectant or excipient may be a non-naturally occurring substance or a naturally occurring substance, but is not limited thereto. In another specific embodiment, the cryoprotectant or excipient may be a substance with which the Enterococcus faecalis EF-2001strain does not naturally come into contact, or a substance that is not naturally included together with the strain, but is not limited thereto.

In another specific embodiment, the composition may further include at least one cryoprotectant selected from the group consisting of glycerol, trehalose, maltodextrin, skim milk powder, and starch; and/or at least one excipient selected from the group consisting of glucose, dextrin, and skim milk.

The cryoprotectant of the present invention may be included in an amount of 0.01 wt% to 20 wt% or 0.01 wt% to 10 wt%, based on the total weight of the composition. Specifically, glycerol may be included in the composition in an amount of 5 wt% to 20 wt%, trehalose in an amount of 2 wt% to 10 wt%, maltodextrin in an amount of 2 wt% to 10 wt%, skim milk powder in an amount of 0.5 wt% to 2 wt%, and starch in an amount of 0.1 wt% to 1 wt%. In addition, the excipient may be included in an amount of 75 wt% to 95 wt% or 85 wt% to 95 wt%, based on the total weight of the composition.

In addition, a method for preparing a composition comprising the Enterococcus faecalis EF-2001 strain, a lysate thereof, a culture thereof, a concentrate thereof, a dried product thereof, or dead cells thereof may include mixing an additive with at least one selected from the group consisting of the Enterococcus faecalis EF-2001 strain, a lysate thereof, a culture thereof, a concentrate thereof, a dried product thereof, and dead cells thereof. The additive may be the above-described cryoprotectant or excipient.

The composition may be a cosmetic composition, a food composition, a health functional food composition, a food additive composition, an external preparation composition, or a pharmaceutical composition.

The Enterococcus faecalis strain, a lysate thereof, a culture thereof, a concentrate thereof, a dried product thereof, or dead cells thereof contained in the composition for improving skin function of the present invention has excellent effects of inhibiting melanin production, enhancing skin elasticity, improving skin barrier function, and soothing the skin, and thus may be usefully used in a cosmetic product, a health functional food, a food additive, an external preparation, or a pharmaceutical composition for improving skin function.

In one embodiment of the present invention, the present invention provides a cosmetic product or external preparation comprising, as an active ingredient, at least one selected from the group consisting of the Enterococcus faecalis strain, a lysate thereof, a culture thereof, a concentrate thereof, a dried product thereof, and dead cells thereof.

In addition, since the methods for obtaining the Enterococcus faecalis strain, a lysate thereof, a culture thereof, a concentrate thereof, a dried product thereof, or dead cells thereof are the same as those described in the composition for improving skin function, the specific description thereof is incorporated by reference, and only configurations specific to the external preparation and cosmetic product will be described below.

The components included in the cosmetic product or external preparation of the present invention may include, in addition to the active ingredient, components commonly used in cosmetic products or external preparations, for example, conventional auxiliary agents such as antioxidants, stabilizers, solubilizers, vitamins, pigments, and fragrances, and carriers.

The active ingredient of the present invention may be easily formulated by carrying out formulation according to conventional methods, and surfactants, excipients, colorants, flavoring agents, preservatives, stabilizers, buffers, suspending agents, and other commonly used auxiliary agents may be suitably used.

The cosmetic product or external preparation of the present invention may be prepared in any formulation commonly prepared in the art, and may be formulated, for example, as a solution, suspension, emulsion, paste, gel, cream, lotion, powder, soap, surfactant-containing cleansing product, oil, powder foundation, emulsion foundation, wax foundation, pack, massage cream, spray, and the like, but is not limited thereto. More specifically, the cosmetic product or external preparation may be prepared in the form of a skin softener, nourishing lotion, nourishing cream, massage cream, essence, eye cream, cleansing cream, cleansing foam, cleansing water, pack, spray, or powder.

When the formulation of the present invention is a paste, cream, or gel, animal oil, vegetable oil, wax, paraffin, starch, tragacanth, cellulose derivatives, polyethylene glycol, silicone, bentonite, silica, talc, zinc oxide, or the like may be used as a carrier component.

When the formulation of the present invention is a solution or emulsion, a solvent, solubilizer, or emulsifier may be used as a carrier component, and examples thereof include water, ethanol, isopropanol, ethyl carbonate, ethyl acetate, benzyl alcohol, benzyl benzoate, propylene glycol, 1,3-butylene glycol oil, glycerol aliphatic acid esters, polyethylene glycol, and sorbitan fatty acid esters.

When the formulation of the present invention is a suspension, liquid diluents such as water, ethanol, or propylene glycol; suspending agents such as ethoxylated isostearyl alcohol, polyoxyethylene sorbitol esters, and polyoxyethylene sorbitan esters; microcrystalline cellulose, aluminum metahydroxide, bentonite, agar, tragacanth, or the like may be used as a carrier component.

When the formulation of the present invention is a powder or spray, lactose, talc, silica, aluminum hydroxide, calcium silicate, polyamide powder, or the like may be used as a carrier component, and particularly in the case of a spray, the formulation may further include a propellant such as chlorofluorohydrocarbon, propane/butane, or dimethyl ether.

In addition, in one embodiment of the present invention, the present invention provides a health functional food or food additive comprising, as an active ingredient, at least one selected from the group consisting of the Enterococcus faecalis strain, a lysate thereof, a culture thereof, a concentrate thereof, a dried product thereof, and dead cells thereof.

Since the methods for obtaining the Enterococcus faecalis strain, a culture solution thereof, and dead cells thereof are the same as those described in the composition comprising, as an active ingredient, the Enterococcus faecalis strain, a culture solution thereof, or dead cells thereof, the specific description thereof is incorporated by reference, and only configurations specific to the health functional food will be described below.

There is no particular limitation on the type of food to which the Enterococcus faecalis of the present invention is added. Examples of the food include drinks, meats, sausages, bread, biscuits, rice cakes, chocolate, candies, snacks, confectionery, pizza, ramen, other noodles, gums, dairy products including ice creams, various soups, beverages, alcoholic beverages, vitamin complexes, and the like, and include all health foods and health functional foods in the ordinary sense.

The mixing amount of the Enterococcus faecalis according to the present invention may be suitably determined depending on the intended use thereof, such as prevention or improvement. In general, the amount of the Enterococcus faecalis in the health food may be added in an amount of 0.001 wt% to 50 wt% based on the total weight of the food. However, in the case of long-term intake for health and hygiene purposes or for health regulation purposes, the amount may be below the above range, and since there is no problem in terms of safety, the active ingredient may also be used in an amount exceeding the above range.

The health functional composition of the present invention is not particularly limited in terms of other components, except that it contains the Enterococcus faecalis as an essential component at the indicated ratio, and may contain various flavoring agents, natural carbohydrates, and the like as additional components, as in ordinary beverages. Examples of the natural carbohydrates described above include conventional sugars such as monosaccharides, for example, glucose and fructose; disaccharides, for example, maltose and sucrose; and polysaccharides, for example, dextrin and cyclodextrin; and sugar alcohols such as xylitol, sorbitol, and erythritol. As flavoring agents other than those described above, natural flavoring agents, such as thaumatin and stevia extract, for example, rebaudioside A and glycyrrhizin, and synthetic flavoring agents, such as saccharin and aspartame, may be advantageously used.

In addition to the above, the food or food additive of the present invention may contain various nutrients, vitamins, minerals (electrolytes), flavoring agents such as synthetic flavoring agents and natural flavoring agents, colorants and enhancers such as cheese and chocolate, pectic acid and salts thereof, alginic acid and salts thereof, organic acids, protective colloidal thickeners, pH regulators, stabilizers, preservatives, glycerin, alcohol, carbonating agents used in carbonated beverages, and the like. In addition, the Enterococcus faecalis of the present invention may contain fruit pulp for preparing natural fruit juices, fruit juice beverages, and vegetable beverages. These components may be used independently or in combination. The proportion of such additives is not particularly critical, but is generally selected within a range of 0 to about 20 parts by weight based on 100 parts by weight of the Enterococcus faecalis of the present invention.

In addition, in one embodiment of the present invention, the present invention provides a pharmaceutical composition comprising, as an active ingredient, at least one selected from the group consisting of the Enterococcus faecalis strain, a lysate thereof, a culture thereof, a concentrate thereof, a dried product thereof, and dead cells thereof.

Since the methods for obtaining the Enterococcus faecalis strain, a lysate thereof, a culture thereof, a concentrate thereof, a dried product thereof, or dead cells thereof are the same as those described in the composition for improving skin function, the specific description thereof is incorporated by reference.

The composition of the present invention may comprise, as an active ingredient, the strain in an amount of 10⁶ to 10¹³ cfu/g based on the total weight of the composition, or may comprise a lysate, culture, concentrate, dried product, or dead cells having an equivalent number of viable cells. In addition, an effective amount of the Enterococcus faecalis strain of the present invention, a culture solution thereof, or dead cells thereof, as a main component, may be mixed with one or two or more pharmaceutically acceptable conventional carriers or one or two or more additives selected therefrom, thereby preparing the composition in a conventional formulation.

As the carrier, one or two or more selected from diluents, lubricants, binders, disintegrants, sweeteners, stabilizers, and preservatives may be used, and as the additive, one or two or more selected from fragrances, vitamins, and antioxidants may be used.

In the present invention, any pharmaceutically acceptable carrier and additive may be used. Specifically, as a diluent, lactose monohydrate, trehalose, cornstarch, soybean oil, microcrystalline cellulose, or D-mannitol is preferred; as a lubricant, magnesium stearate or talc is preferred; and as a binder, polyvinyl pyrrolidone (PVP) or hydroxypropylcellulose (HPC) is preferred. In addition, as a disintegrant, carboxymethylcellulose calcium (Ca-CMC), sodium starchglycolate, polacrylin potassium, or cross-linked polyvinylpyrrolidone is preferred; as a sweetener, one selected from sucrose, fructose, sorbitol, or aspartame is used; as a stabilizer, one selected from carboxymethylcellulose sodium (Na-CMC), beta-cyclodextrin, white bee's wax, or xanthan gum is used; and as a preservative, one selected from methyl p-hydroxybenzoate (methylparaben), propyl p-hydroxybenzoate (propylparaben), or potassium sorbate is preferred, but is not limited thereto.

The pharmaceutical composition may be administered to a patient as a single dose, or may be administered by a fractionated treatment protocol in which multiple doses are administered over a long period of time. As used herein, the term "pharmaceutically effective amount" refers to an amount that produces a response greater than that of a negative control, and preferably refers to an amount sufficient to prevent or treat an inflammatory disease. In addition, the pharmaceutically effective amount may be appropriately varied depending on various factors such as the disease and its severity, the age, weight, health condition, sex, route of administration, and duration of treatment of the patient.

The composition of the present invention may be formulated in various ways according to the route of administration by a method known in the art together with the pharmaceutically acceptable carrier. As used herein, the term "pharmaceutically acceptable" refers to a non-toxic composition that is physiologically acceptable and, when administered to humans, does not inhibit the action of the active ingredient and does not typically cause allergic reactions such as gastrointestinal disorders and dizziness, or similar reactions. The composition of the present invention may be formulated in various ways according to the route of administration by a method known in the art together with the pharmaceutically acceptable carrier. The route of administration is not limited thereto, but may be oral or parenteral.

Hereinafter, the present invention will be described in more detail through examples. It will be apparent to those skilled in the art that the following examples are presented only as examples to describe the present invention in more detail, and the scope of the present invention is not to be construed as being limited by these examples.

### Preparation Example 1. Preparation of a Culture Solution of Enterococcus faecalis EF-2001

Live cells of Enterococcus faecalis EF-2001 (Korea Verm Co., Ltd., Korea) were aerobically or anaerobically cultured in a medium generally used for culturing lactic acid bacteria, followed by seed culture, and then main culture was performed for 1 to 3 days while maintaining a pH of 5.0 to 8.0 and a temperature of 20°C to 40°C, so as to reach a bacterial cell count of 7.5 × 10¹² cfu/g or more based on dry weight (DW).

Thereafter, centrifugation was performed at 8,000 rpm for 10 minutes to separate a supernatant. The separated supernatant was diluted 10-fold (10%, v/v) to prepare a culture solution of Enterococcus faecalis EF-2001.

### Preparation Example 2. Preparation of Dead Cells of Enterococcus faecalis EF-2001

The Enterococcus faecalis EF-2001cultured in Preparation Example 1 was heat-treated at 70°C to 130°C for 5 minutes to 30 minutes to be killed, and then bacterial cells were separated and recovered using a continuous centrifuge, followed by freeze-drying and powdering.

### Experimental Example 1. Evaluation of Primary Cilia Activation

### 1-1. Preparation and Culture of Cell Lines

Human fibroblasts from skin (GM00969, Corriell Institute), keratinocytes (HaCaT), and B16F1 cells, which are a mouse-derived melanoma cell line (mouse melanoma, ATCC), were cultured in DMEM (Dulbecco’s modified Eagle’s medium) supplemented with 15% or 10% (v/v) fetal bovine serum (FBS) and 1% penicillin-streptomycin (Invitrogen, CA, USA) under conditions of 37°C in a 5% (v/v) CO₂ incubator.

### 1-2. Treatment with Enterococcus faecalis EF-2001

In order to confirm activation of primary cilia formation by EF-2001 treatment in the cell lines, cells were cultured in a 12-well culture plate (SPL Life Sciences, South Korea) at a cell number of 8 × 10⁴ cells per well for 24 hours. From the time when the cells reached about 90%, the cells were treated for 24 hours with a control containing only medium, a stress-inducing substance, particulate matter 2.5 (PM2.5; 30 μg/ml), alone, or media containing each concentration of test substances, namely a culture solution of EF-2001 (0.1, 1, and 5%) and dead cells of EF-2001 (0.01, 0.1, and 1 mg/ml), together with the stress-inducing substance PM2.5.

### 1-3. Evaluation of Primary Cilia Activation of Enterococcus faecalis EF-2001

In order to confirm the primary cilia activation effect of EF-2001, human fibroblasts from skin were treated for 24 hours with cell culture media containing, at each concentration, a simple medium in which EF-2001 was not cultured, a culture solution of EF-2001 (indicated as “Sub.”), and dead cells of EF-2001 (indicated as “Bacteria”), alone or together with the stress-inducing substance PM2.5 (30 μg/ml). Thereafter, immunostaining was performed using an antibody against ARL13B (17711-1-AP, 1:1000 dilution, Proteintech, Chicago, IL), which is a major marker of primary cilia, and Hoechst dye for nuclear staining, and a primary cilia formation ratio and the length of primary cilia were measured using a fluorescence microscope (IX71, Olympus, Japan).

Here, the primary cilia formation ratio was expressed as a percentage of the number of cells having primary cilia relative to the total number of cells, and the length of primary cilia was obtained by measuring the actual length of primary cilia observed under a microscope.

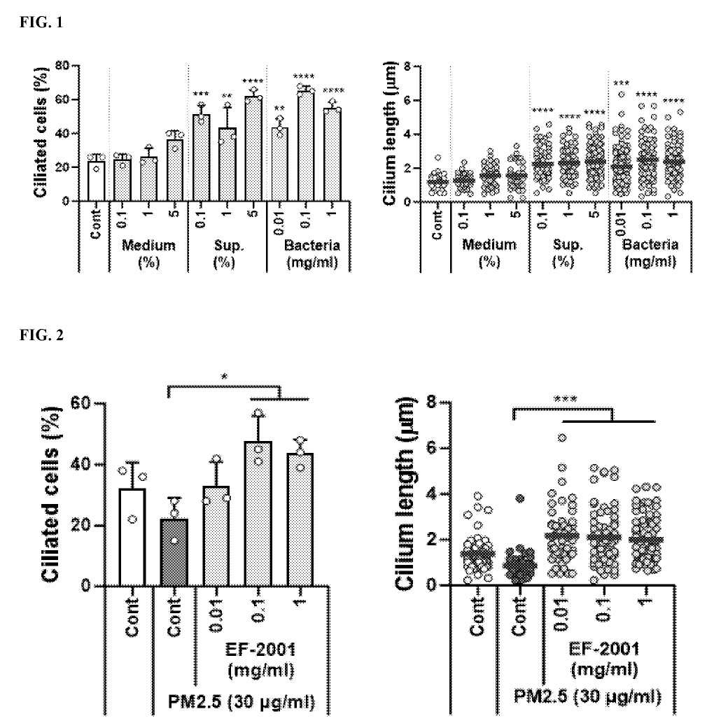

As a result, as shown in FIG. 1, it was confirmed that treatment with the culture solution and dead cells of EF-2001 alone at each concentration increased primary cilia activity. In addition, when the stress-inducing substance, namely fine dust PM2.5, was treated, primary cilia formation was inhibited. However, when dead cells of EF-2001 were treated together with the stress-inducing substance PM2.5, it was confirmed that primary cilia formation was statistically significantly restored to a normal level or higher, as shown in FIG. 2 (**p < 0.01, ***p < 0.005, ****p < 0.001).

### Experimental Example 2. Evaluation of Melanin Production Inhibitory Effect

The mouse-derived melanoma cell line B16F1 (ATCC, USA) cells cultured as described above were pretreated with 0.5 μM of a melanin-producing hormone, α-MSH (alpha-melanocyte-stimulating hormone, Sigma-Aldrich), for 48 hours, and then further treated with the culture solution of EF-2001 according to Preparation Example 1 (0.1, 1, and 5%) and the dead cells according to Preparation Example 2 (0.01, 0.1, and 1 mg/ml) for 24 hours, followed by measurement of melanin content. Here, the melanin content was analyzed by dissolving the prepared cells in an aqueous NaOH solution and then measuring absorbance.

At this time, melasolv (10 ppm) was used as a positive control substance for a whitening effect through inhibition of melanin production. The melanin content was analyzed as a percentage relative to a control group by dissolving the treated B16F1 cells in an aqueous NaOH solution and then measuring absorbance at a wavelength of 475 nm using a spectrophotometer.

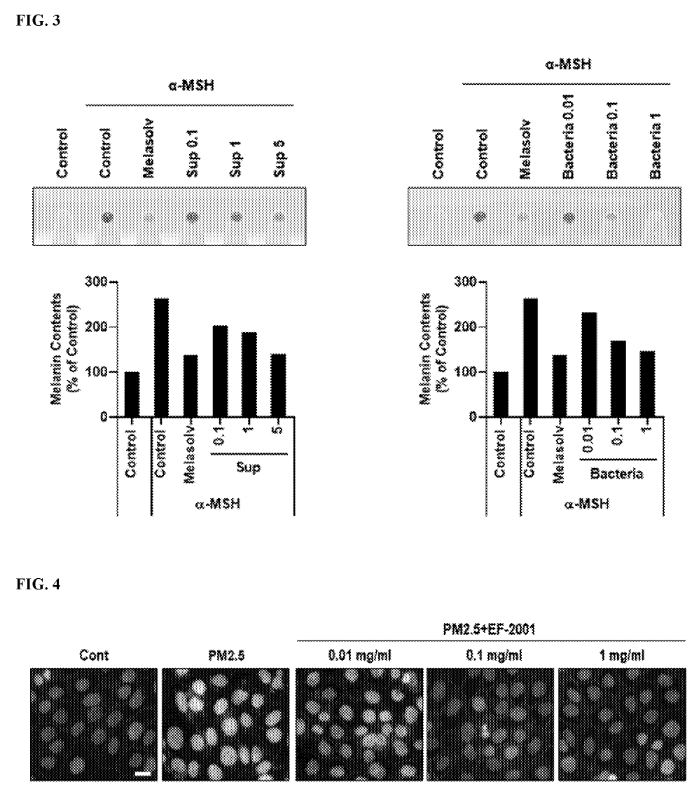

As a result, as shown in FIG. 3, it was confirmed that the amount of melanin production, which was increased about three-fold by α-MSH, which is a melanin-producing hormone, was remarkably inhibited under treatment conditions with the culture solution of EF-2001 and dead cells of EF-2001. Accordingly, it was confirmed that the culture solution of EF-2001 and dead cells of EF-2001 according to the present invention are useful for skin whitening.

### Experimental Example 3. Evaluation of Improvement of Skin Barrier Function

A damaged skin barrier is considered to be a major risk factor for skin sensitization, and among them, Claudin-1 is known as an essential factor for the barrier function of the epidermis.

In order to evaluate the effect of EF-2001 according to the present invention on improvement of the skin barrier, human-derived keratinocytes were treated together with the stress-inducing substance PM2.5 ( 30 μg/ml) and dead cells of EF-2001 at each concentration (0.01, 0.1, and 1 mg/ml), and after 24 hours, expression of Claudin-1 (#37-4900, 1:1000 dilution, Thermo-Fisher), which is a major membrane protein of the epithelial barrier, was confirmed by fluorescent immunostaining.

Specifically, all samples prepared from the cells were dissolved in 2× Laemmli sample buffer [62.5 mM Tris-HCl, pH 6.8, 25% (v/v) glycerol, 2% (w/v) sodium dodecyl sulfate (SDS), 5% (v/v) β-mercaptoethanol, and 0.01% (w/v) bromophenol blue (Bio-Rad, Hercules, CA, USA)] for western blotting. All cellular proteins were quantified using Bradford solution (Bio-Rad) according to the manufacturer’s instructions. Thereafter, the samples were separated by sodium dodecyl sulfate-polyacrylamide gel electrophoresis (SDS-PAGE), transferred to a polyvinylidene fluoride membrane (PVDF, Bio-Rad), blocked with 4% (w/v) skim milk in Tris-buffered saline and Tween [TBST; 25 mM Tris, 140 mM sodium chloride, and 0.05% (v/v) Tween 20], and then reacted overnight at 4°C with the following specific primary antibodies:
actin (ACTA1; 1:10,000 dilution, MAB1501, Millipore, MA, USA), Claudin-1 (1:3,000 dilution, #37-4900, Thermo Fisher, MA, USA), phospho-NF-κB p65 (1:1,000 dilution, #3031, Cell Signaling Technology, MA, USA), and MMP-1 (1:3,000 dilution, NB600-1192, Novous Biologicals, CO, USA).

For protein detection, the samples were reacted with a horseradish peroxidase (HRP)-conjugated secondary antibody (Cell Signaling Technology) at room temperature for 2 hours, and luminescence signals were measured using Clarity Western ECL substrate (ATTO, Moto Asakusa, Tokyo, Japan).

The expression level of each protein was confirmed using an AE-9300 Ez-Capture MG Hours Image Saver HR image capture tool (WSE-7120L, ATTO, Tokyo, Japan).

The treated human-derived keratinocytes were fixed with 4% (w/v) paraformaldehyde, permeabilized with phosphate-buffered saline (PBS) containing 0.1% (v/v) Triton X-100 (PBS-T), blocked with 1% (v/v) bovine serum albumin (BSA), and then incubated overnight at 4°C with a primary antibody against Claudin-1 (#37-4900, 1:1000 dilution, Thermo-Fisher), which is an important marker substance for the cell barrier. Thereafter, after washing with PBS, Alexa Fluor 555 goat anti-mouse (Invitrogen), diluted 1:1000 with the PBS, was used as a secondary antibody and incubated at room temperature for 2 hours. Cell nuclei were stained with 1 μg/ml Hoechst33342 dye (H3570, Thermo-Fisher) at room temperature for 20 minutes, and barrier formation images were obtained using a fluorescence microscope IX71 (IX71, Olympus, Japan).

As a result, as shown in FIG. 4, when the stress-inducing substance PM2.5 was treated, expression of Claudin-1 was clearly reduced and the barrier was abnormally formed. In contrast, when dead cells of EF-2001 according to the present invention were added, it was confirmed that barrier formation was noticeably improved together with an increase in Claudin-1 fluorescence expression.

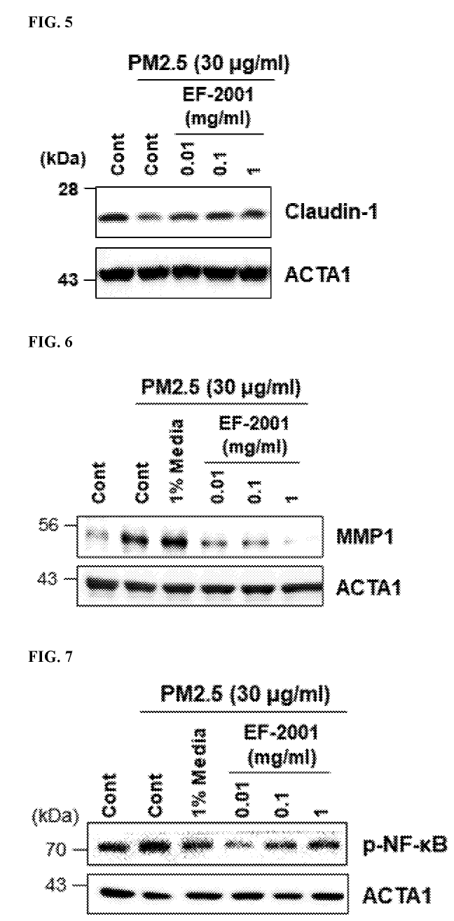

In addition, this tendency was also confirmed by western blotting. As shown in FIG. 5, it was confirmed that expression of Claudin-1 protein, which was reduced by the stress-inducing substance PM2.5, was restored by treatment with dead cells of EF-2001. Considering these results, it can be confirmed that EF-2001 according to the present invention is effective in improving skin barrier function.

### Experimental Example 4. Evaluation of Skin Wrinkle Improvement and Anti-inflammatory Effects

The human-derived skin fibroblasts were treated with dead cells of EF-2001 at each concentration (0.01, 0.1, and 1 mg/ml) together with the stress-inducing substance PM2.5 (30 μg/ml), and after 24 hours, a cell lysate was prepared. After protein quantification, protein changes were measured by western blotting using a matrix metalloproteinase-1 (MMP-1)-specific antibody.

As a result, as shown in FIG. 6, when the cells were treated with the stress-inducing substance PM2.5 (30 μg/ml), expression of MMP-1, which is a collagenase, increased compared to the control group. In contrast, when dead cells of EF-2001 according to the present invention were treated, it was confirmed that the expression of MMP-1 increased by the stress-inducing substance PM2.5 was reduced to the level of the control group.

In addition, since nuclear factor-κB (NF-κB) p65 is known as a major inflammatory transcription factor that regulates inflammatory responses, the effect of EF-2001 according to the present invention on activation of NF-κB in cells treated with the stress-inducing substance PM2.5 was additionally confirmed.

As a result, as shown in FIG. 7, when dead cells of EF-2001 according to the present invention were treated, it was confirmed that activation of NF-κB by the stress-inducing substance PM2.5 was inhibited in human-derived fibroblasts.

Taken together, these results suggest that EF-2001 according to the present invention may have wrinkle improvement and soothing effects by activating formation of primary cilia in fibroblasts, thereby inhibiting expression of collagenase and activation of inflammatory factors increased by the stress-inducing substance PM2.5.

From the above description, a person having ordinary skill in the art to which the present invention pertains will understand that the present invention may be embodied in other specific forms without changing the technical spirit or essential features thereof. In this regard, the embodiments described above should be understood as illustrative in all respects and not restrictive. The scope of the present invention should be construed such that all changes or modifications derived from the meaning and scope of the claims described below and equivalent concepts thereof, rather than from the detailed description above, are included within the scope of the present invention.

1. A composition for improving skin function, comprising, as an active ingredient, at least one selected from the group consisting of an Enterococcus faecalis strain, a lysate thereof, a culture thereof, a concentrate thereof, a dried product thereof, and dead cells thereof.

2. The composition for improving skin function according to claim 1, wherein the strain is an Enterococcus faecalis EF-2001 strain.

3. The composition for improving skin function according to claim 1, wherein the composition activates primary cilia.

4. The composition for improving skin function according to claim 1, wherein the composition inhibits melanin production.

5. The composition for improving skin function according to claim 1, wherein the composition enhances skin elasticity.

6. The composition for improving skin function according to claim 1, wherein the composition has activity of improving skin barrier function.

7. The composition for improving skin function according to claim 1, wherein the composition has a skin soothing effect.

8. A cosmetic product for improving skin function, comprising, as an active ingredient, at least one selected from the group consisting of an Enterococcus faecalis strain, a lysate thereof, a culture thereof, a concentrate thereof, a dried product thereof, and dead cells thereof.

9. A health functional food for improving skin function, comprising, as an active ingredient, at least one selected from the group consisting of an Enterococcus faecalis strain, a lysate thereof, a culture thereof, a concentrate thereof, a dried product thereof, and dead cells thereof.

10. A food additive for improving skin function, comprising, as an active ingredient, at least one selected from the group consisting of an Enterococcus faecalis strain, a lysate thereof, a culture thereof, a concentrate thereof, a dried product thereof, and dead cells thereof.

11. An external preparation for improving skin function, comprising, as an active ingredient, at least one selected from the group consisting of an Enterococcus faecalis strain, a lysate thereof, a culture thereof, a concentrate thereof, a dried product thereof, and dead cells thereof.

12. A pharmaceutical composition for improving skin function, comprising, as an active ingredient, at least one selected from the group consisting of an Enterococcus faecalis strain, a lysate thereof, a culture thereof, a concentrate thereof, a dried product thereof, and dead cells thereof.
